(19) United States
(12) Patent Application Publication
(10) Pub. No.: US 2008/0146525 A1
(43) Pub. Date: Jun. 19, 2008

(54) COMPOSITION FOR PROMOTING COGNITIVE ATTRIBUTES

(75) Inventors: Marvin A. Heuer, Mississauga (CA); Shan Chaudhuri, Brampton (CA); Ken Clement, Mississauga (CA)

Correspondence Address: TORYS LLP, 79 WELLINGTON ST. WEST, SUITE 3000, TORONTO, ON, M5K 1N2, omitted

(73) Assignee: H3 FORMULATIONS LTD., Mississauga (CA)

(21) Appl. No.: 11/945,562

(22) Filed: Nov. 27, 2007

Related U.S. Application Data
(60) Provisional application No. 60/868,852, filed on Dec. 6, 2006.

(51) Int. Cl. A61K 31/661; A61K 31/495; A61P 25/00

(52) U.S. Cl. 514/114; 514/256

(57) ABSTRACT

A nutritional composition and method is provided for enhancing cognition, providing nootropic effects to improve concentration in an individual by supporting the biological activity of the neurotransmitters comprising therapeutically effective amounts of Sulbutiamine or derivatives thereof, an effective amount of Huperzine-A or derivatives thereof and an effective amount of Tyrosine or derivatives thereof.

### CROSS REFERENCE TO RELATED APPLICATIONS

This application claims the benefit of U.S. Provisional Patent Application Ser. No. 60/868,852, filed Dec. 6, 2006, the content of which is incorporated by reference.

### FIELD OF THE INVENTION

The present invention relates to a nutritional composition and method for enhancing cognition, and providing nootropic effects to improve concentration and mental focus during athletic performance.

### BACKGROUND OF THE INVENTION

The term “nootropic” was first termed in 1972 by C. E. Guirgea in relation to substances employed for the enhancement of learning and memory (Balaraman R, Shingala J. The molecule of the millennium: Nootropics. Indian Journal of Pharmacology 2002; 34: 439-440). The main features associated with the effects of nootropics include the following: enhancement of learning and memory acquisitions as well as resistance of learned behaviors to agents that impair them; protection of the brain against physical or chemical injuries; facilitation of interhemispheric flow of information and efficient tonic cortical/subcortical mechanisms; and an absence of the negative effects.

A large portion of research on nootropics has been directed at deriving compositions for the treatment of conditions such as Alzheimer's disease, epilepsy and stroke. Despite efforts, a common nootropic mechanism has not been found (Gualtieri F, Manetti D, Romanelli M N, Ghelardini C. Design and study of piracetam-like nootropics, controversial members of the problematic class of cognition-enhancing drugs. Curr Pharm Des. 2002;8(2):125-38) however most putative nootropics exert some effect on either neurotransmitters or associated receptors. Neurotransmitters are molecules that trigger changes in electrical potential in the synapses of neurons to allow signal transduction along nerves.

Signaling via the neurotransmitter acetylcholine is intimately involved in memory, attention and learning (Kuczewski N, Aztiria E, Gautam D, Wess J, Domenici L. Acetylcholine modulates cortical synaptic transmission via different muscarinic receptors, as studied with receptor knockout mice. J Physiol. Aug. 1, 2005 (Pt 3):907-19). Loss of acetylchoine neurotransmission capacity has been associated with many neurodegenerative diseases such as Parkinson's disease (Fujita M, Ichise M, Zoghbi S S, Liow J S, Ghose S, Vines D C, Sangare J, Lu J Q, Cropley V L, Iida H, Kim K M, Cohen R M, Bara-Jimenez W, Ravina B, Innis R B. Widespread decrease of nicotinic acetylcholine receptors in Parkinson's disease. Ann Neurol. 2006 Jan;59(1):174-7). Acetylcholine-based neurotransmission is complex, with elaborate feedback mechanisms and receptor subtypes that respond to various stimuli. Acetylcholine is synthesized by the enzyme choline acetyltransferase from choline and acetyl-coenzymeA. Memory tasks induce the release of acetylcholine in the brain and can be augmented by a number of factors including glucose (Ragozzino M E, Unick K E, Gold P E. Hippocampal acetylcholine release during memory testing in rats: augmentation by glucose. Proc Natl Acad Sci U S A. May 14, 1996;93(10):4693-8). Glucose serves as a precursor of acetyl-coenzymeA.

Dopamine is itself a neurotransmitter that also serves as a precursor for other neurotransmitters, namely epinephrine (adrenaline) and norepinephrine (noradrenaline). Dopamine plays a role in memory and as with the case in acetylcholine neurotransmission, reduced dopamine-related neurotransmission is associated with neurodegeneration (Fujita M, Ichise M, Zoghbi S S, Liow J S, Ghose S, Vines D C, Sangare J, Lu J Q, Cropley V L, lida H, Kim K M, Cohen R M, Bara-Jimenez W, Ravina B, Innis R B. Widespread decrease of nicotinic acetylcholine receptors in Parkinson's disease. Ann Neurol. 2006 Jan;59(1):174-7), neurodevelopment (Wong D F, Harris J C, Naidu S, Yokoi F, Marenco S, Dannals R F, Ravert H T, Yaster M, Evans A, Rousset O, Bryan R N, Gjedde A, Kuhar M J, Breese G R. Dopamine transporters are markedly reduced in Lesch-Nyhan disease in vivo. Proc Natl Acad Sci U S A. May 28, 1996;93(11):5539-43) and psychiatric conditions (Kwak Y T, Koo M S, Choi C H, Sunwoo I. Change of dopamine receptor mRNA expression in lymphocyte of schizophrenic patients. BMC Med Genet. 2001;2:3). Dopamine control of neurotransmission is regulated by complex processes involving feedback mechanisms (Paladini C A, Robinson S, Morikawa H, Williams J T, Palmiter R D. Dopamine controls the firing pattern of dopamine neurons via a network feedback mechanism. Proc Natl Acad Sci U S A. Mar. 4, 2003;100(5):2866-71). Data suggests that dopamine-related neurotransmission is important in and is increased during many cognitive tasks (Fried I, Wilson C L, Morrow J W, Cameron K A, Behnke E D, Ackerson L C, Maidment N T. Increased dopamine release in the human amygdala during performance of cognitive tasks. Nat Neurosci. Feb. 4, 2001;4(2):201-6). As such neurotransmitters are important in learning and cognition as well as in neuroprotective mechanisms.

It is advantageous to promote cognitive attributes, particularly during times of strenuous activity such as heavy resistance training, when blood supply ,and therefore nutrients are typically shunted away from the brain to active skeletal muscle (Delp M D, O'Leary D S. Integrative control of the skeletal muscle microcirculation in the maintenance of arterial pressure during exercise. J Appl Physiol. 2004 Sep;97(3):1112-8).

### SUMMARY OF THE INVENTION

The foregoing needs and other needs and objectives that will become apparent for the following description are achieved in the present invention, which comprises a nutritional supplement and method directed towards enhancing cognition, and providing nootropic effects to improve and maintain concentration during athletic performance or strenuous workouts. The nutritional supplement comprises and effective combination of Sulbutiamine or derivatives thereof, Huperzine-A or derivatives thereof, and the amino acid Tyrosine or derivatives thereof.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional supplement for enhancing cognition, and providing nootropic effects to improve and maintain concentration during athletic performance or strenuous workouts in a mammal.

It is herein understood that the terms “enhancing cognition” and “nootropic effects” jointly refer to factors that affect facets of brain function including, but not limited to memory, mental focus, learning, mental performance, concentration and attention.

It is also herein understood that since cognitive attributes depend upon the proper physical conditions, integrity and function of both the central and peripheral nervous systems, any substance that promotes the health of the central and peripheral nervous systems and constituents thereof e.g. neurons, glia, microglia, satellite cells, oligodendrocytes, etc., is considered to be a substance which enhances cognition, and provides nootropic effects. Such substances also include substances which promote the protection and proper functioning of the central and peripheral nervous systems including neurotransmitters, growth factors, the cells and mechanisms that synthesize and secrete said neurotransmitters and growth factors as well as the cells and mechanisms which express the receptors for said neurotransmitters and growth factors.

In a preferred embodiment of the present invention, a composition comprising at least an effective amount of Sulbutiamine or derivatives thereof, an effective amount of Huperzine-A or derivatives thereof and an effective amount of Tyrosine or derivatives thereof is provided.

In another embodiment of the present invention, a composition comprising an effective amount of Sulbutiamine or derivatives thereof and an effective amount of Huperzine-A or derivatives thereof and one or more of the following: Vinpocetine, Alpha-glycerophosphocholine and Cis-9, 10-octadecenoamide or respective derivatives thereof is provided.

### Sulbutiamine

Sulbutiamine is a precursor to thiamine (vitamin B1) and is often used to treat fatigue. It has been shown to have memory enhancing effects in rats (Bizot J C, Herpin A, Pothion S, Pirot S, Trovero F, Ollat H. Chronic treatment with sulbutiamine improves memory in an object recognition task and reduces some amnesic effects of dizocilpine in a spatial delayed-non-match-to-sample task. Prog Neuropsychopharmacol Biol Psychiatry. 2005 Jul;29(6):928-35 Abstract). The effects of Sulbutiamine may be due to the modulation of dopaminergic and glutamatergic binding sites in specific regions of the brain (Trovero F, Gobbi M, Weil-Fuggaza J, Besson M J, Brochet D, Pirot S. Evidence for a modulatory effect of sulbutiamine on glutamatergic and dopaminergic cortical transmissions in the rat brain. Neurosci Lett. Sep. 29, 2000; 292(1):49-53 Abstract.

In an embodiment of the present invention, which is set forth in greater detail in the example below, the nutritional supplement includes Sulbutiamine or derivatives thereof. A serving of the nutritional supplement may include from about 0.0005 g to about 0.005 g of Sulbutiamine or derivatives thereof. The preferred dosage of a serving of the nutritional supplement comprises about 0.001 g of Sulbutiamine or derivatives thereof.

### Huperzine-A

Huperzine-A is an alkaloid derived from the Chinese herb Huperzia serrata plant. It is a potent inhibitor of acetylcholinesterase which catalyzes the hydrolysis, or breakdown, of the neurotransmitter acetylcholine. Acetylcholine has various effect in different cell types, Different cell types are programmed and equipped to response to the same signal in different ways (Molecular Biology of the Cell, 3^{rd} Edition. 1994. Bruce Alberts, Dennis Bray, Julian Lewis, Martin Raff, Keith Roberts, and James D. Watson. Garland Publishing, pg 726-728). Thus, two different cell types, may respond to acetylcholine such that different outward responses are observed. Indeed, acetylcholine signaling in the sensory cortex is important for sensory-cognitive function (Metherate R. Nicotinic acetylcholine receptors in sensory cortex. Learn Mem. 2004 Jan-Feb;11(1):50-9) and also plays a role in muscle contraction (Molecular Biology of the Cell, 3^{rd} Edition. 1994. Bruce Alberts, Dennis Bray, Julian Lewis, Martin Raff, Keith Roberts, and James D. Watson. Garland Publishing, pg 540).

Moreover, Huperzine-A has been shown to have similar effects to compositions commonly used to treat Alzheimer's disease, however with a greater ability to increase extra-cellular acetylcholine and dopamine levels (Liang Y Q, Tang X C. Comparative studies of huperzine A, donepezil, and rivastigmine on brain acetylcholine, dopamine, norepinephrine, and 5-hydroxytryptamine levels in freely-moving rats. Acta Pharmacol Sin. 2006 Sep;27(9):1127-36 Text only). Animal model experiments and experiments in humans indicate that Huperzine-A improves memory deficits and is safe. Furthermore when administered orally, huperzine-A shows good bioavailability (Wang R, Yan H, Tang X C. Progress in studies of huperzine A, a natural cholinesterase inhibitor from Chinese herbal medicine. Acta Pharmacol Sin. 2006 Jan;27(1):1-26 Text only). In addition to its good bioavailability, Huperzine-A has also been shown to improve memory and learning in health adolescents (Sun Q Q, Xu S S, Pan J L, Guo H M, Cao W Q. Huperzine-A capsules enhance memory and learning performance in 34 pairs of matched adolescent students. Zhongguo Yao Li Xue Bao. 1999 Jul;20(7):601-3). Mechanistically distinct from it's effect on acetylcholine, Huperzine-A has also been shown to have the ability to increase the amount of nerve growth factor. This growth factor is important for nervous system development and function, as well as protection against various types of neuronal injury. (Tang L L, Wang R, Tang X C. Effects of huperzine A on secretion of nerve growth factor in cultured rat cortical astrocytes and neurite outgrowth in rat PC12 cells. Acta Pharmacol Sin. 2005 Jun;26(6):673-8 Text only).

In an embodiment of the present invention, which is set forth in greater detail in the example below, the nutritional supplement includes Huperzine-A or derivatives thereof. A serving of the nutritional supplement may include from about 0.01 mg to about 0.09 mg of Huperzine-A or derivatives thereof. The preferred dosage of a serving of the nutritional supplement comprises about 0.05 mg of Huperzine-A or derivatives thereof.

### Tyrosine

The amino acid Tyrosine has nootropic activity by virtue of being a precursor of the neurotransmitter, dopamine. Oral administration of Tyrosine to humans results in increased of neurotransmitters, indicative of increased neurotransmitter synthesis in the peripheral nervous system (Agharanya J C, Alonso R, Wurtman R J. Changes in catecholamine excretion after short-term tyrosine ingestion in normally fed human subjects. Am J Clin Nutr. 1981 Jan;34(1):82-7 Abstract). This suggests that oral Tyrosine that crossing the blood-brain barrier is capable of increasing neurotransmitter synthesis in the brain. Tyrosine is used in the synthesis of dopamine first via hydration by the enzyme tyrosine hydroxlyase to form DOPA, then by decarboxylation by the enzyme aromatic-L-amino-acid decarboxylase.

In an embodiment of the present invention, which is set forth in greater detail in the example below, the nutritional supplement includes Tyrosine or derivatives thereof. A serving of the nutritional supplement may include from about 0.500 g to about 1.500 g of Tyrosine or derivatives thereof. The preferred dosage of a serving of the nutritional supplement comprises about 1.010 g of Tyrosine or derivatives thereof.

Not wishing to be bound by theory, it is understood by the inventors that the composition of the present invention, when administered to an individual enhances cognition, and provides nootropic effects. In particular, administration to an individual acts to improve and maintain concentration, particularly during athletic performance or strenuous workouts.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is as a powder.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients or be a functional component of a broader composition, as is well known in the art.

In various embodiments of the present invention, the composition or portion thereof is provided in the form of fine-milled particles. As used herein, the terms “fine-milled” and/or “fine-milling” refer the process of micronization. Micronization is a mechanical process which involves the application of force to a particle, thereby resulting in a reduction in the size of said particle. U.S. Provisional Application No.: 60/776,325 entitled “Compositions and Method for Increasing Bioavailability of Compositions for Performance Improvement”, discloses a method of improving the absorption, palatability, taste, texture and bioavailability of compounds by increasing the solubility. The increased bioavailability of a compound or ingredients is achieved via a reduction in particle size using a “fine-milling” technique. Any acceptable fine-milling technique wherein the result is the fine-milled particles having an average particle size of between about 50 microns to about 2 microns is appropriate for the purposes of this disclosure. The reduction in size of the particles increases the surface area-to-volume ratio of each particle, thus increasing the rate of dissolution, thereby improving the rate of absorption.

The present nutritional composition or those similarly envisioned by one of skill in the art, may be utilized in methods promote to enhance cognition, and provide nootropic effects to improve and maintain concentration during athletic performance or strenuous workouts.

Although the following example illustrates the practice of the present invention in one of its embodiments, the example should not be construed as limiting the scope of the invention. Other embodiments will be apparent to one of skill in the art from consideration of the specifications and example.

### EXAMPLE

A nutritional supplement is provided for daily administration in powdered form. A single serving of the nutritional supplement comprises from about 0.0005 g to about 0.005 g of Sulbutiamine, from about 0.01 mg to about 0.09 mg of Huperzine-A and from about 0.500 g to about 1.500 g of Tyrosine.

Directions: As a nutritional supplement, one serving of said powder is mixed with 8 oz. of water and consumed daily. Each serving is preferably consumed within 45 minutes prior to exercise.

What is claimed:

1. A composition for enhancing cognition, and providing nootropic effects to improve and maintain concentration in a mammal comprising:
an effective amount of Sulbutiamine or derivatives thereof, an effective amount of Huperzine-A or derivatives thereof and an effective amount of Tyrosine or derivatives thereof,
whereby the components of said composition act jointly and simultaneously support nervous system integrity and function.

2. A method for enhancing cognition, and providing nootropic effects to improve and maintain concentration in a mammal comprising:
administering an effective amount of Sulbutiamine or derivatives thereof, an effective amount of Huperzine-A or derivatives thereof and an effective amount of Tyrosine or derivatives thereof,
whereby the components of said composition act jointly and simultaneously support nervous system integrity and function.

3. The composition of claim 1 further comprising one or more of the following:
Vinpocetine or derivatives thereof, Alpha-glycerophosphocholine or derivatives thereof and Cis-9, 10-octadecenoamide or derivatives thereof.

4. The method of claim 2 further comprising one or more of the following:
Vinpocetine or derivatives thereof, Alpha-glycerophosphocholine or derivatives thereof and Cis-9, 10-octadecenoamide or derivatives thereof.
